(19) United States
(12) Patent Application Publication
(10) Pub. No.: US 2026/0249066 A1
(43) Pub. Date: Aug. 27, 2026

(54) COMPRESSION VALVE

(71) Applicant: Merit Medical Systems, Inc., South Jordan, UT (US)

(72) Inventors: Michael Dean Haslam, Sandy, UT (US); Nicole Willardson, Herriman, UT (US); Richard D. Jenkins, Kennett Square, PA (US)

(21) Appl. No.: 19/547,910

(22) Filed: Feb. 24, 2026

Related U.S. Application Data
(60) Provisional application No. 63/762,958, filed on Feb. 25, 2025.

(51) Int. Cl. A61M 39/06; A61M 25/06

(52) U.S. Cl. CPC A61M 39/0613; A61M 2025/0681

(57) ABSTRACT

A compression valve is provided. The compression valve can include a housing including an engagement portion including a cross-sectional area that is substantially elliptical or rectangular, a valve member disposed within the housing, the valve member including a lumen, and a compression member proximally attached to the housing. The compression member is configured to compress the valve member against the engagement portion such that a cross-sectional area of the lumen of the valve member is substantially elliptical or rectangular.

### RELATED APPLICATIONS

This application claims priority to United States Provisional Application No. 63/762,958, filed on February 25, 2025 and titled, “Compression Valve,” which is hereby incorporated by reference in its entirety.

### TECHNICAL FIELD

The present application relates to stent prostheses, guidewires, catheters and methods of using the same. More particularly, some embodiments relate to a compression hemostasis valve.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1A illustrates a side view of an introducer system, according to embodiments described herein.

FIG. 1B illustrates an end-on view of a compression valve of the introducer system of FIG. 1A, according to embodiments described herein.

FIG. 2 illustrates a perspective view of the compression valve of FIGS. 1A-B, according to embodiments described herein.

FIG. 3 illustrates a perspective, exploded view of the compression valve of FIGS. 1A-B, according to embodiments described herein.

FIG. 4 illustrates a perspective, cutaway view of the housing of the compression valve of FIG. 2, according to embodiments described herein

FIG. 5 illustrates an overhead view of the housing of the compression valve of FIG. 2, according to embodiments described herein.

FIG. 6A illustrates a perspective, cutaway view of the housing of FIG. 5 taken through line 6A-6A, according to embodiments described herein.

FIG. 6B illustrates a perspective, cutaway view of the housing of FIG. 5 taken through line 6B-6B, according to embodiments described herein.

FIG. 7A illustrates a lateral cutaway view of the valve of FIGS. 1A-B in an open configuration, according to embodiments described herein.

FIG. 7B illustrates a lateral cutaway view of the valve of FIGS. 1A-B in a closed configuration, according to embodiments described herein.

FIG. 8 illustrates a perspective view of an embodiment of a valve member configured to be used in a compression valve, according to embodiments described herein.

FIG. 9A illustrates a perspective, cutaway view of the valve of FIG. 8 in a compression valve, according to embodiments described herein.

FIG. 9B illustrates a perspective, cutaway view of the valve of FIG. 8 in a compression valve, according to embodiments described herein.

FIG. 10A illustrates a lateral, cutaway view of the valve of FIG. 8 in an open configuration, according to embodiments described herein.

FIG. 10B illustrates a lateral, cutaway view of the valve of FIG. 8 in a closed configuration, according to embodiments described herein.

FIG. 11 illustrates a perspective view of an embodiment of a valve member configured to be used in a compression valve, according to embodiments described herein.

FIG. 12 illustrates a perspective view of an embodiment of a valve member configured to be used in a compression valve, according to embodiments described herein.

FIG. 13 illustrates a perspective view of an embodiment of a valve member configured to be used in a compression valve, according to embodiments described herein.

FIG. 14 illustrates a perspective view of an embodiment of a valve member configured to be used in a compression valve, according to embodiments described herein.

FIG. 15 illustrates a perspective view of an embodiment of a valve member configured to be used in a compression valve, according to embodiments described herein.

### DETAILED DESCRIPTION

Introducer sheaths are used in a variety of diagnostic and therapeutic procedures to provide access to a patient’s vascular system. An introducer sheath may be placed into the vasculature of a patient to facilitate the exchange of guidewires, catheters, contrast media, fluids, and so on, while providing access and a hemostatic seal to the vasculature. The introducer sheath can include a proximal hub, or handle, situated externally to the patient's skin, forming a chamber accessible through various ports like a side port and an introducer bore. For example, the side port can fluidly connect to a channel controlled by a stop-cock, forming a fluidic path to the patient’s vasculature. Guidewires, catheters, stents, and other tools can be introduced through the bore into the patient’s vasculature. A valve can be used to ensure hemostasis while allowing the insertion of medical instruments.

The seal maintained by the valve can impact the safety and effectiveness of a procedure. The seal may be configured to be airtight and fluid tight and/or limit the air or fluid that passes the valve. Limiting fluid flow across the seal may result in increased safety during a procedure as the seal formed by a valve around a medical tool can serve as a barrier against the potential leakage of fluids from the vascular system, prevent blood loss, and/or reduce the risk of contamination or infection. A seal around an instrument can also enhance control over fluid flow, allowing for the accurate administration of fluid (e.g., medications or contrast agents) during therapeutic procedures.

To maintain a seal, the material of a valve or valve member can be elastic or compressible. This property can allow it to closely conform to the shape of a device (e.g., a guidewire) that is passed through the valve and maintain a secure closure around the device. For instance, as a guidewire is inserted into the introducer chamber of a valve, the valve member can tightly close around the guidewire, effectively sealing the entry point.

In some procedures, multiple devices (e.g., guidewires, catheters, etc.) may need to be introduced through the same valve and introducer bore. For instance, in interventional procedures like angioplasty or stent placement, healthcare practitioners may sequentially introduce different guidewires or catheters (through the introducer and/or valve) to access different branches of the vasculature or to perform specific maneuvers. Use of one valve and/or introducer bore can streamline a procedure by minimizing the number of incisions, or access points, used.

One issue with conventional introducers, is that they can struggle or fail to adequately maintain a full seal during such use cases where multiple devices are introduced through the same valve member. For instance, the insertion of multiple and separate, devices through a valve member can create varied geometries, as well as opportunities for gaps or irregularities to form in the seal. A conventional valve member may struggle to fit and maintain a tight seal around the shape created by such devices. Moreover, varying, or asymmetric, sizes and shapes of different devices can increase the challenges in forming a uniform and effective seal. This can allow fluid to escape, or air to enter the vascular system.

Systems and methods of the present disclosure address the above and other challenges by providing a valve and valve member configured to maintain a seal against multiple medical devices inserted through an introducer. In some embodiments, the valve and valve member of the introducer can include an ovular or elliptical sealing surface that can enhance the integrity of the hermitic seal when multiple devices are introduced. In some embodiments, the valve and valve member of the introducer can induce medical devices passing through the valve member to arrange sequentially or lineally. In some embodiments, the valve and valve member of the introducer can form a seal against the sequential or lineal configuration of the devices.

The components of the embodiments as generally described and illustrated in the figures herein can be arranged and designed in a wide variety of different configurations. Thus, the following more detailed description of various embodiments, as represented in the figures, is not intended to limit the scope of the present disclosure, but is merely representative of various embodiments. While various aspects of the embodiments are presented in drawings, the drawings are not necessarily drawn to scale unless specifically indicated.

The phrase “coupled to” is broad enough to refer to any suitable coupling or other form of interaction between two or more entities, including mechanical, fluidic and/or thermal interaction. Thus, two components may be coupled to each other even though they are not in direct contact with each other. The phrases “attached to” or “attached directly to” refer to interaction between two or more entities which are in direct contact with each other and/or are separated from each other only by a fastener of any suitable variety (e.g., mounting hardware or an adhesive). The phrase “fluid communication” is used in its ordinary sense, and is broad enough to refer to arrangements in which a fluid (e.g., a gas or a liquid) can flow from one element to another element when the elements are in fluid communication with each other.

The terms “proximal” and “distal” are opposite directional terms. For example, the distal end of a device or component is the end of the component that is furthest from the practitioner during ordinary use. The proximal end refers to the opposite end, or the end nearest the practitioner during ordinary use.

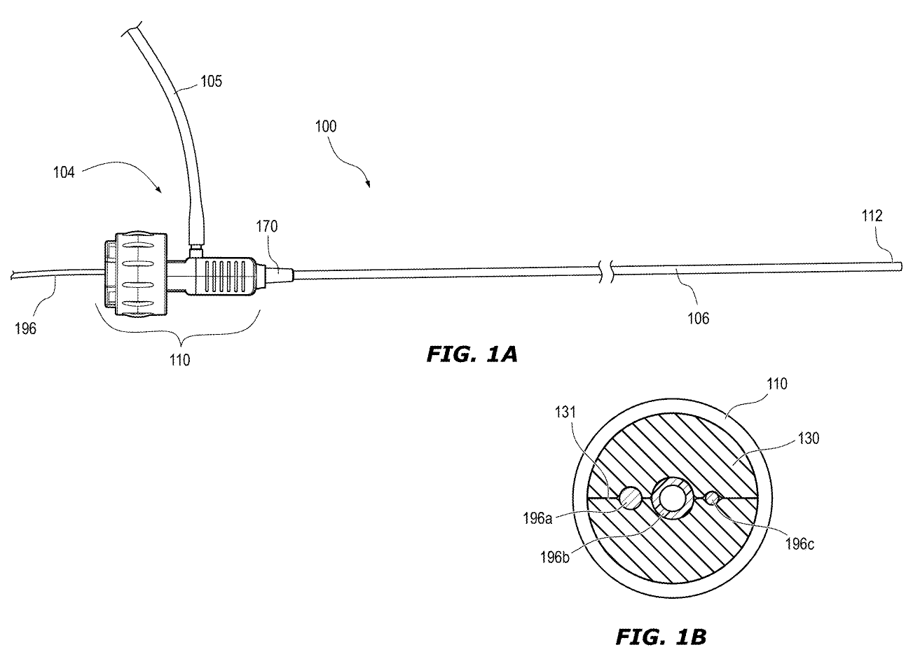

FIG. 1A illustrates a side view of an introducer system 100, according to embodiments disclosed herein. Introducer system 100 can include a proximal portion 104, including a compression valve 110 fluidly connected to a sheath 106 (leading to a distal end 112).

The sheath 106 can be fluidly connected to a distal side of the proximal portion 104. In some embodiments, the sheath 106 can extend distally from a distal end 170 of the proximal portion 104, to a distal end 112, or delivery end, of the introducer system 100. The proximal portion 104 can provide a proximal user input, with one or more components configured to allow a practitioner to deploy or otherwise manipulate further devices (e.g., such as a guidewire 196) disposed within the introducer system 100.

During use, the proximal portion 104 can be disposed outside of a patient’s body, while the sheath 106 can be inserted into a patient’s vasculature. In some embodiments, the sheath 106 may extend a short distance into a patient’s vasculature, past an insertion site. In alternate embodiments, the introducer system 100 can be a catheter system, and the sheath 106 can be advanced to a treatment location within the patient’s body.

In some embodiments, the proximal portion 104 can include the compression valve 110 which may form a fluidic seal around one or more medical devices passing through the valve. (In some embodiments, the compression valve 110 can be a part of a larger handle assembly, within the proximal portion 104). The proximal portion 104 and the compression valve 110 can further include a secondary fluid passageway 105 fluidly connected to a main fluid passageway of the proximal portion 104.

The compression valve 110 of the proximal portion 104 can be used to lock, close, or seal, against the flow of fluid around one or more elongate members (e.g., such as the guidewire 196) passing through a fluid passageway of the compression valve 110. For instance, in some cases, one, two, or any number of elongate members (e.g., guide wires, catheters, etc.) can be passed through a proximal end of the compression valve 110, through the fluid passageway, and out the sheath 106 into the vasculature of a patient.

Thus, the compression valve 110 can be employed by a user of the introducer system 100 to close and maintain a seal against such elongate members, and otherwise maintain hemostasis. When in use, the compression valve 110 can provide a fluid seal around one or more elongate members passing through compression valve 110 and into the vasculature of a patient.

FIG. 1B illustrates an end-on view of multiple elongate members 196A-C passing through the compression valve 110 of the introducer system 100 of FIG. 1A, according to embodiments described herein. In the illustrated embodiment, a valve member 130 of the compression valve 110 that is deformable forms a seal (illustrated by line 131) against the three elongate members 196A-C, by collapsing around the exterior circumferences of the one or more elongate members. In some cases, the valve member 130 can be formed from multiple components, in others, the valve member 130 can be a monolithic piece.

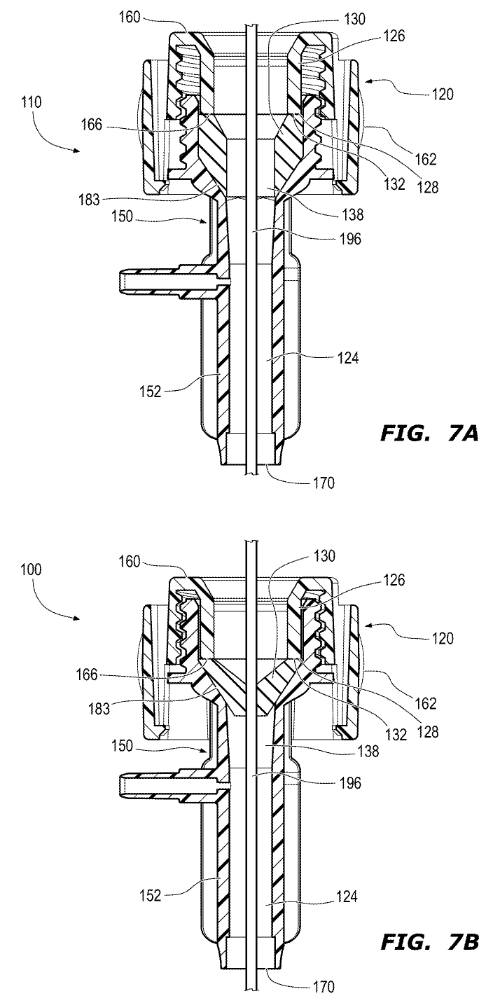

In some embodiments, the valve member 130 of the compression valve 110 can deform in such a way that any elongate members passing through the compression valve 110 become disposed sequentially. While FIG. 1B illustrates the three elongate members 196A-C disposed sequentially without contact each other, the three elongate members 196A-C may be placed in a variety of different configurations, such as in direct contact with each other or forming a triangular shape, and the like. The seal formed against each of the elongate members 196A-C can be maintained within a closed configuration of the compression valve 110, which will be further discussed with respect to FIGS. 7A-7B.

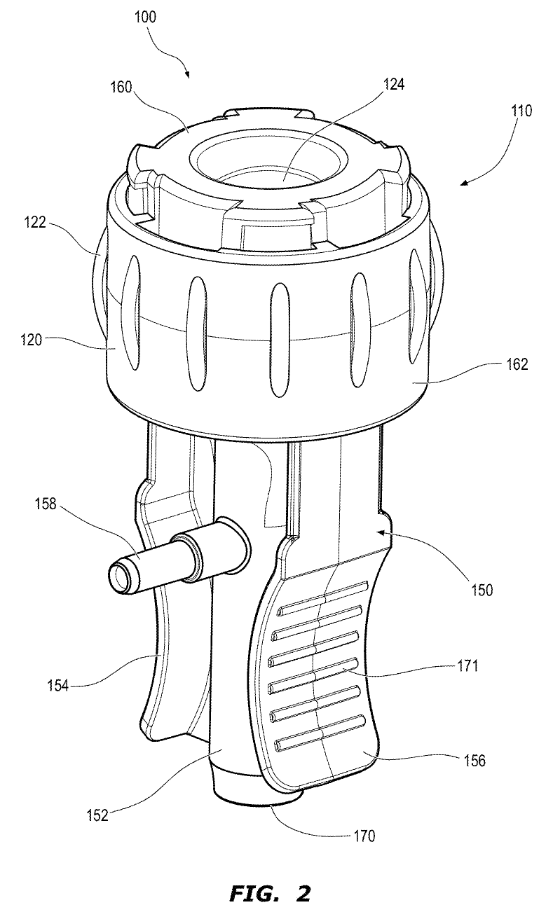

FIG. 2 illustrates a perspective view of the compression valve 110 of the introducer system 100 of FIGS. 1A-B, according to embodiments described herein. In the illustrated embodiment, the compression valve 110 includes a compression member 120 and a housing 150.

In some embodiments, one or more medical devices can be introduced into compression valve 110 through a cavity or passageway (e.g., passageway 124) of the compression valve 110. The passageway 124 can extend through a longitudinal axis (vertically with respect to the page) of the compression valve 110. The passageway 124 can be defined by at least by a portion of the compression member 120 and the housing 150, and extend to a distal end 170 of the compression valve 110.

The compression member 120 can include a stationary portion 160 and a rotating portion 162. The rotating portion 162 can rest exterior to the stationary portion 160, and be configured to rotate with respect to both the stationary portion 160 and the housing 150. The stationary portion 160 can be configured to be non-rotating, or stationary, with respect to the rotating portion 162. The rotating portion 162 can include ridges 122 to facilitate gripping or grasping by an operator of the compression valve 110. The compression valve 110 can be operated i.e., engage or disengage a seal against one or more medical devices within the passageway 124, by rotating the rotating portion 162 of compression member 120 with respect to the stationary portion 160 and the housing 150.

The housing 150 can include a connecting portion (not seen in FIG. 2) for fluidly connecting to the compression member 120 and an elongate portion 152, defining an internal bore or cavity and extending longitudinally with respect to the compression valve 110. The housing 150 can further include a port or vent (e.g., vent 158) for exhausting fluid or relieving pressure from the elongate portion 152.

In some embodiments, the housing 150 can also include grasping portions 154, 156 for gripping the housing 150. In some embodiments, the grasping portions 154, 156 can extend longitudinally down one or more sides of the housing 150. The grasping portions 154, 156 can include ridges 171 for facilitating grasping or gripping by an operator of the compression valve 110.

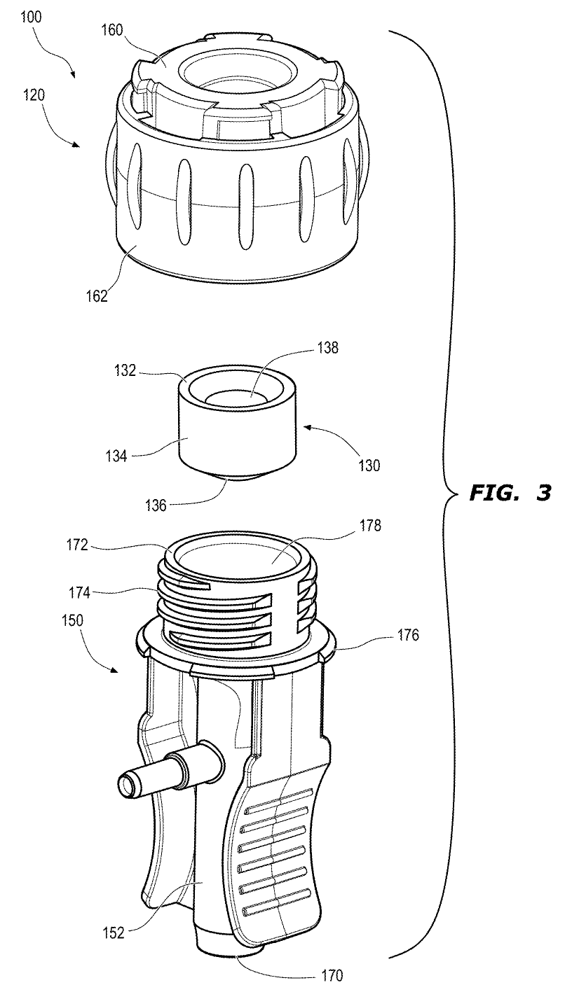

FIG. 3 illustrates a perspective, exploded view of a valve of the introducer system 100 of FIGS. 1A-B, according to embodiments described herein. As seen in the illustrated embodiment, a valve member 130 can be configured to be disposed within a receptacle 178 of the housing 150. In some embodiments the valve member 130 can be situated between the compression member 120 and the housing 150.

In some embodiments, the valve member 130 can include a proximal portion 132 and an outer wall 134 extending from the proximal portion 132 to a distal face 136. A lumen 138 can extend through the valve member 130. In some embodiments, the lumen 138 can form a portion of the passageway 124 of the compression valve 110.

The valve member 130 can be made of a deformable, and/or elastomeric material. For example, the valve member 130 can be configured to be compressed, and the lumen 138 can be configured to deform, collapse, or otherwise tighten constricting the diameter of the lumen 138. For example, due to such deformation, the diameter of the lumen 138 can constrict, or tighten, around one or more medical devices passing through the lumen 138. In some embodiments, the valve member 130 can be symmetrical about an axis of the lumen 138.

The housing 150 can include a connection portion 172. The connection portion 172 can be configured to mate with the compression member 120. In some embodiments, threads 174 can be used to mate the housing 150 and the compression member 120. A ridge 176 can be configured to arrest the threading or mating of the housing 150 and the compression member 120, after a certain amount of threading. The receptacle 178 can be configured to receive the valve member 130.

To operate the valve, the rotating portion 162 can be rotated with respect to the housing 150. For example, to close the valve around one or more elongate devices passing through the compression valve 110, the rotating portion 162 can be rotated in a first direction with respect to the housing 150. To open the valve, the rotating portion 162 can be rotated in a second, or opposite, direction.

As the rotating portion 162 is rotated with respect to the housing 150, the valve member 130 can undergo a compressive, or deforming, force in the longitudinal direction of the valve. As the valve member 130 becomes compressed, the valve member 130 lumen 138 can collapse or become constricted. Otherwise stated, the diameter of lumen 138 can diminish.

An inner geometry of the surfaces within the receptacle 178 can influence the geometry of the collapsed, or collapsing, lumen 138. The lumen 138 can be influenced to resemble various shapes or geometries. For instance, in some embodiments, as the valve member 130 deforms, and the lumen 138 becomes constricted, a cross-sectional shape of the lumen 138 can become a circle, an oval, an ellipse, a rectangle, etc., that diminishes in size. In some embodiments, the cross-sectional shape of the lumen 138 can continue to diminish in size until a seal is formed around one or more elongate medical devices passing through the compression valve 110.

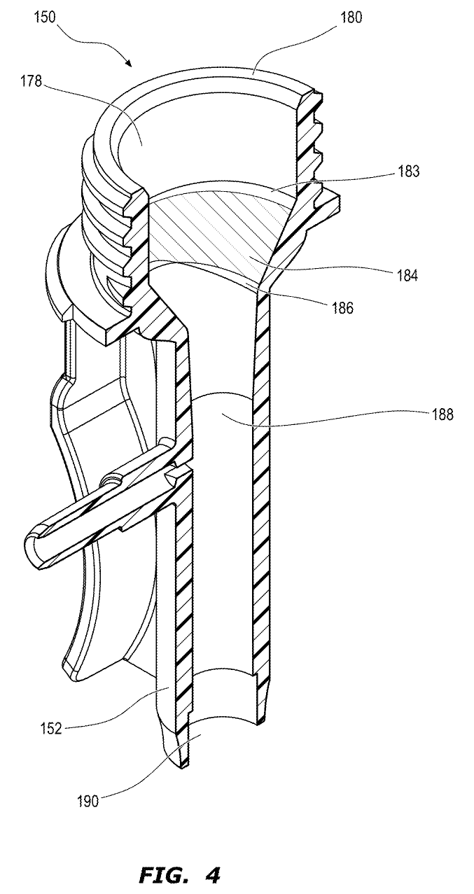

FIG. 4 illustrates a perspective, cutaway view of the housing 150 of the compression valve 110 of FIG. 2, according to embodiments described herein. In the illustrated embodiment, the receptacle 178 is disposed at, and/or includes, an interior portion of the housing 150. In some embodiments, the receptacle 178 can be configured to receive and engage with the valve member 130 (not shown). The receptacle 178 can include specific geometries configured to deform or compress the valve member 130 upon compression via the compression member 120 against varying surfaces of the receptacle 178.

The receptacle 178 can include a proximal portion 180 defining a proximal end of the receptacle 178. An inner cavity wall 182 can extend longitudinally from proximal portion 180 to a valve member engagement portion 183. The valve member engagement portion 183 can have a substantially conical surface. In some embodiments, the valve member engagement portion 183 include a first conical portion 184 and a second conical portion 186. The first conical portion 184 can extend into the second conical portion 186. The receptacle 178 can have a distal portion 188, which can be a distal end of receptacle 178. Distal portion 188 can transition the receptacle 178 to a lumen 190 of the elongate portion 152 of the housing 150.

The first conical portion 184 can include a surface that is conical, or similar to a cone. For example, the first conical portion 184 can be or include a surface that tapers smoothly from a circular base to a smaller, ovular, or elliptical base. For example, the first conical portion 184 can be characterized as a curved surface which extends from the circular base or cross-section to a smaller, elliptical base or cross-section, forming a single continuous curve. A cross-section of first conical portion 184 from a proximal end to a distal end can begin as a circle and (moving in the distal direction of the valve) transition to an ellipse.

The second conical portion 186 can extend from the first conical portion 184, and can similarly be configured to engage with a lower surface of the valve member 130 (not shown). In some embodiments, the second conical portion 186 can include a surface that is conical, or similar to a cone, with an elliptical or ovular cross-section. For example, the second conical portion 186 can be or include a surface that tapers smoothly from a larger elliptical base to a smaller elliptical base. For example, the second conical portion 186 can be characterized as a curved surface which extends from a larger ovular base to a smaller ovular base, forming a single continuous curve. A cross-section of second conical portion 186 can be an oval or ellipsis that transitions to a smaller oval or ellipses (moving in the distal direction of the valve). In some embodiments, the second conical portion 186 can transition to a circular base (moving in the distal direction of the valve).

The inner cavity defined by the valve member engagement portion 183 can decrease in overall cross-sectional area (moving from a proximal end to a distal end of the valve member engagement portion). In some embodiments, the first conical portion 184 can transition from a circular base or cross-sectional area (at the proximal end) to an elliptical base or cross-sectional area (at the distal end). The second conical portion 186 can transition from an elliptical base or cross-sectional area (at the proximal end) to a smaller elliptical base or cross-sectional area.

In some embodiments, as the valve member 130 (not shown) is compressed against the valve member engagement portion 183, the lumen 138 of the valve member 130 can be influenced to have a cross-section that is ovular, elliptical, or rectangular in shape. Thus, the lumen 138 of the valve member 130 may have a cross-sectional shape that transitions from a first (e.g., initial), circular shape, to an intermediate ovular shape, to a final slit, or sealing shape (as seen in FIG. 1B). Furthermore, the overall size of the lumen 138 and cross-sectional area of the lumen 138 can diminish as the valve member 130 is compressed against the valve member engagement portion 183.

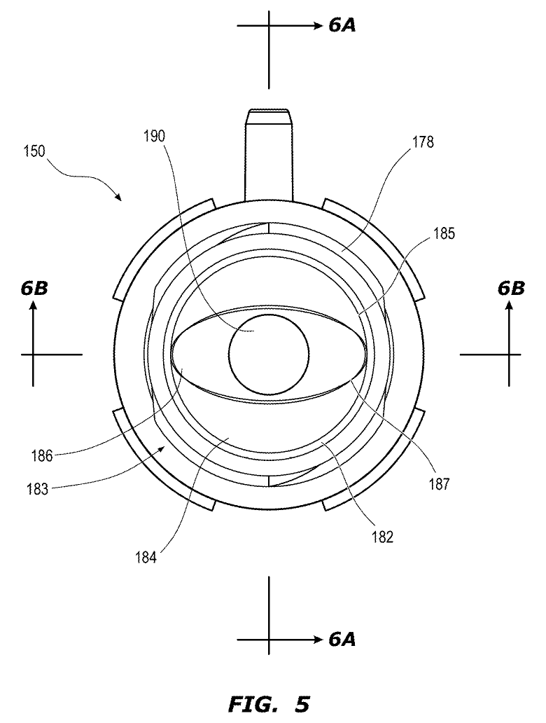

FIG. 5 illustrates an overhead view of the housing 150 of the compression valve 110 of FIG. 2, according to embodiments described herein. In the illustrated embodiment, the receptacle 178 and the engagement portion 183 can be seen, including the first conical portion 184, the second conical portion 186, and the lumen 190.

The first conical portion 184 can transition from a circular base 185 of the inner cavity wall 182, to the elliptical base 187, of the second conical portion 186. The second conical portion 186 can transition from the elliptical base 187, to the circular base 185, or cross-section of lumen 190.

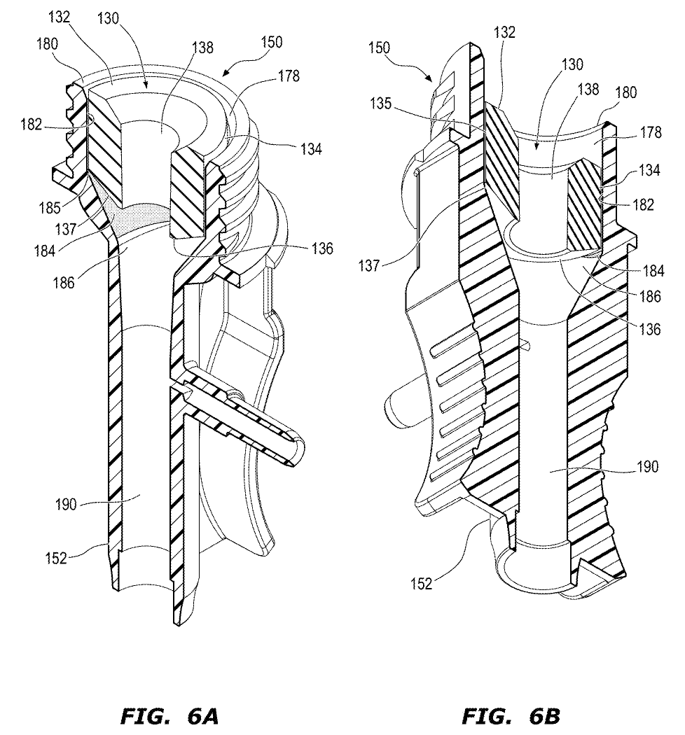

FIGS. 6A-B illustrate perspective, cutaway views of the valve member 130 seated within housing 150 (of FIGS. 2 and 5), and will be described in tandem. Accordingly, FIG. 6A illustrates a perspective, cutaway view of the housing of FIG. 5 taken through line 6A-6A, according to embodiments described herein. FIG. 6B illustrates a perspective, cutaway view of the housing of FIG. 5, taken through line 6B-6B, according to embodiments described herein.

In the illustrated embodiment, the valve member 130 is seated within the receptacle 178 of the housing 150. The valve member 130 is seated concentrically within the receptacle 178 such that the outer edge 137 of the distal face 136 of the valve member 130 aligns, or fits concentrically, with the circular base 185 of the inner cavity wall 182. As seen, the distal face 136 extends radially inward and distal, within the cavity formed by the valve member engagement portion 183 (e.g., the first conical portion 184 and the second conical portion 186).

In some embodiments, a first or major diameter of the receptacle 178 of the first conical portion 184 of the valve member engagement portion 183 can remain constant, while a second or minor diameter of the receptacle 178 can diminish (moving distally through the first conical portion 184).

In some embodiments, a first or major diameter of the receptacle 178 of the second conical portion 186 of the valve member engagement portion 183 can diminish (moving in a distal direction), while a second or minor diameter of the second conical portion 186 can remain substantially constant (moving in a distal direction).

In some embodiments, a ratio of the major diameter of the first conical portion 184 of the valve member engagement portion 183 to the minor diameter of the first conical portion 184 of the valve member engagement portion 183 increases along a direction from a proximal end of the valve member engagement portion 183 to a distal end of the valve member engagement portion 183. In some embodiments, a ratio of the major diameter of the second conical portion 186 of the valve member engagement portion 183 to the minor diameter of the second conical portion 186 of the valve member engagement portion 183 increases along a direction from a proximal end of the valve member engagement portion 183 to a distal end of the valve member engagement portion 183.

Accordingly, the compression member 120 is configured to compress the valve member 130 against the valve member engagement portion 183 such that a cross-sectional area of the lumen 138 of the valve member 130 comprises a major diameter and a minor diameter. As the valve member 130 is further compressed, the major diameter increases and the minor diameter decreases.

The valve member 130 is configured to be compressed by the compression member 120 against the valve member engagement portion 183 from an initial shape to an intermediate shape to a final shape. The aspect ratio of the cross-section of the lumen 138 of the valve member 130 changes from the initial shape, to the intermediate shape, to the final shape. For example, the lumen 138 of the valve member 130 is configured to have a circular cross-sectional initial shape. The lumen 138 of the valve member 130 is configured to have an elliptical cross-sectional intermediate shape. The aspect ratio of the intermediate shape is greater than the aspect ratio of the initial shape. The lumen 138 of the valve member 130 is configured to have a rectangular cross-sectional final shape. In other words, the final shape is more or less a slit. The aspect ratio of the final shape is greater than the aspect ratio of the intermediate shape and the initial shape.

The shape of the lumen 138 of the valve member 130 discussed above is determined without medical devices inserted into the lumen 138. The insertion of medical devices in the lumen 138 affects the shape of the lumen 138 of the valve member 130 and insertion of medical devices into the lumen 138 would not have the specific shaped discussed above.

FIGS. 7A-B illustrate lateral, cutaway views of the valve member 130 seated within the housing 150 of the compression valve 110 of FIGS. 1A-B, and will be described in tandem. FIG. 7A illustrates a lateral, cutaway view of the compression valve 110 of FIGS. 1A-B with the valve member 130 in an open configuration, according to embodiments described herein. FIG. 7B illustrates a perspective, cutaway view of the compression valve 110 of FIGS. 1A-B with the valve member 130 in a closed configuration, according to embodiments described herein.

Within the open configuration seen in FIG. 7A, the valve member 130 is seen in an uncompressed state. The lumen 138 of the valve member 130 is open, and the guidewire 196 can pass through the passageway 124 of the compression valve 110.

To transition the compression valve 110 from the open configuration to the closed configuration seen in FIG. 7B, the rotating portion 162 of the compression member 120 can be rotated, with respect to the housing 150. As the rotating portion 162 is rotated, the compression member 120 can translate distally with respect to the housing 150. As the compression member 120 translates distally, an inner edge 166 of the stationary portion 160 can exert a compressive force onto the proximal portion 132 of the valve member 130. The compressive force can cause the valve member 130 to compress, or be compressed against the valve member engagement portion 183 of the housing 150.

As the valve member 130 is compressed against the valve member engagement portion 183, a shape, or profile, of a cross-section of the lumen 138 of valve member 130 can transition from a circular shape to an elliptical shape, or slit (eventually, to a seal as seen in FIG. 1B). As the valve member 130 is further compressed against the valve member engagement portion 183, the cross-section of the lumen 138 of the valve member 130 can transition from the elliptical shape, to a smaller, or diminished elliptical shape. Otherwise stated, a total cross-sectional area of the lumen 138 of valve member 130 can diminish.

In some embodiments, compression of the valve member 130 against the first conical portion 184 of the valve member engagement portion 183 can transition a cross-section of lumen 138 from a circular profile to an elliptical profile. Compression of the valve member 130 against the second conical portion 186 of the valve member engagement portion 183 can transition a cross-section of lumen 138 from the elliptical profile to a smaller elliptical profile. Compression of the valve member 130 against both the first conical portion 184, the second conical portion 186, and/or the valve member engagement portion 183 can diminish a total cross-sectional area of the lumen 138 of valve member 130 completely to a slit.

As seen in FIG. 7B, as the cross-sectional area of the lumen 138 of valve member 130 diminishes, a seal can be formed against the guidewire 196, passing through the lumen 138. In some embodiments, the compression valve 110 can form a seal against one or more, or any number of elongate members passing through the valve (e.g., such as the guidewire 196).

In some embodiments, the compression valve 110 can be transitioned from the closed configuration of FIG. 7B, back to the open configuration of FIG. 7A by rotating the rotating portion 162 such that the compression member 120 is retracted with respect to the housing 150. The valve member 130 can thus return to a relaxed or uncompressed state. The valve member 130 can resiliently return to an uncompressed state; the seal may thus be broken, or disengaged.

In some embodiments, a distal face 128 of an inner wall 126 may be fixed to a proximal portion 132 of the valve member 130. For example, the distal face 128 of the inner wall 126 may be fixed to the proximal portion 132 by an adhesive. Therefore when the compression member 120 is retracted with respect to the housing 150, the connection between the proximal portion 132 of the valve member 130 and the distal face 128 of the inner wall 126 helps return the valve member 130 to the uncompressed state rather than relying on the valve member 130 to just resiliently return to the uncompressed state over time. In other words, the connection causes the valve member 130 to return more quickly to its uncompressed state.

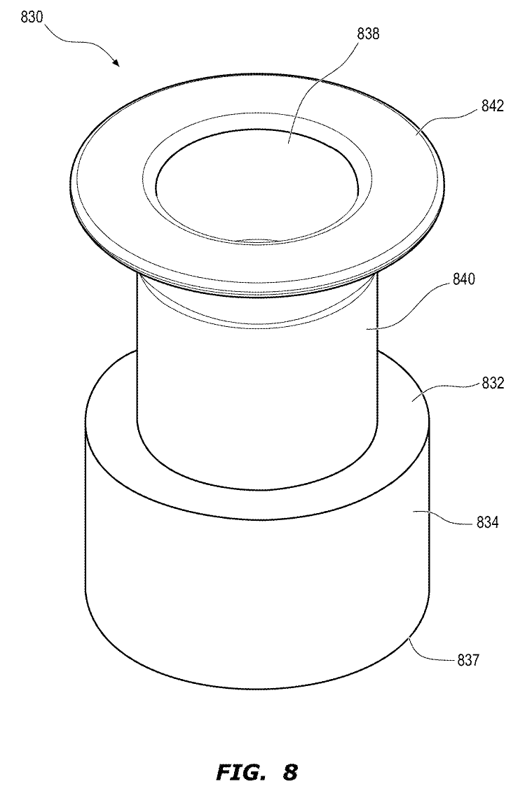

FIGS. 8-15 illustrate embodiments of valve members (and corresponding compression valves) that resemble the valve member (and corresponding compression valves) described with respect to FIGS. 1A-7B, in certain respects. Accordingly, like features are designated with like reference numerals. For example, the embodiments illustrated in FIGS. 8-15 include a housing 150 that may, in some respects, resemble housing 150 of FIGS. 2-7B. Relevant disclosure set forth above regarding similarly identified features thus may not be repeated hereafter. Moreover, specific features of the valve members and corresponding compression valves of FIGS. 8-15, and related components shown in FIGS. 8-15, may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the embodiments of the valve members and corresponding compression and related components illustrated in FIGS. 8-15. Any suitable combination of the features, and variations of the same, described with respect to the valve member, corresponding compression valve, and related components described with respect to FIGS. 1A-7B can be employed with the embodiments the valve members, corresponding compression valves, and related components of FIGS. 8-15, and vice versa. This pattern of disclosure applies equally to further embodiments depicted in subsequent figures and described hereafter, wherein the leading digits may be further incremented.

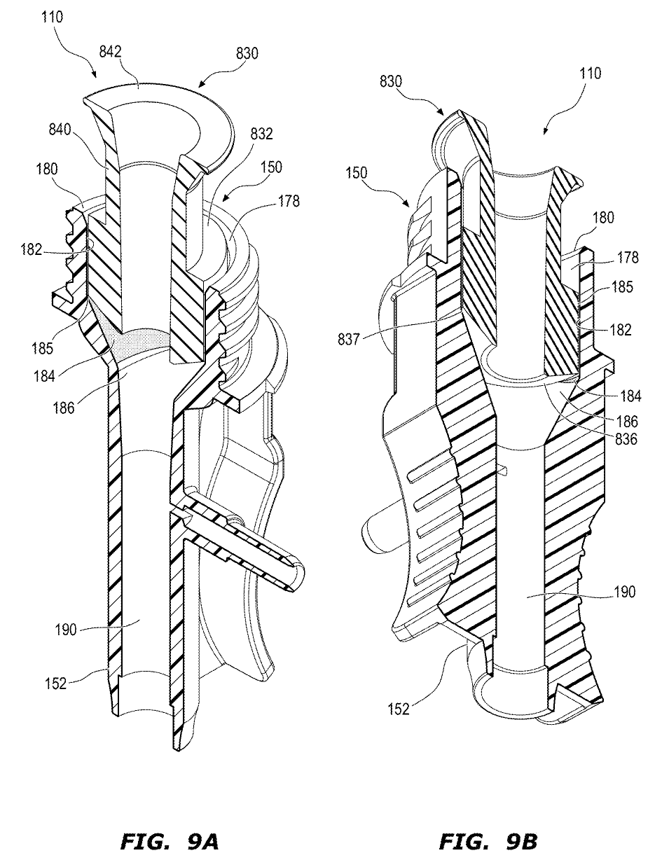

FIG. 8 illustrates a perspective view of an embodiment of a valve member 830 configured to be used with a compression valve 110, according to embodiments described herein. In the illustrated embodiment, the valve member 830 includes a proximal portion 832 and an outer wall 834 extending from the proximal portion 832 to an outer edge 837 of a distal face 836 (see FIG. 9B). A lumen 838 can extend through the valve member 830. In some embodiments, the lumen 838 can form a portion of the passageway 124 of the compression valve 110.

An extension portion 840 can extend from the proximal portion 832 and connect to an anchoring member 842. A face or surface of the anchoring member 842 can be configured to engage with a portion of the compression member 120, and anchor or fix the valve member 830 to the compression member 120. Anchoring of the valve member 830 to the compression member 120 can facilitate the transition from a closed configuration of the valve to an open configuration, by enhancing retraction of the valve member 830 from within the valve member engagement portion 183 of the housing 150.

FIGS. 9A-B illustrate perspective, cutaway views of the embodiment of valve member 830 of FIG. 8, seated within the housing 150 of the compression valve 110 of FIG. 2, and will be described in tandem. Accordingly, FIG. 9A illustrates a perspective, cutaway view of the compression valve 110 of FIG. 2, according to embodiments described herein. FIG. 9B illustrates a perspective, cutaway view of the compression valve 110 of FIG. 2, according to embodiments described herein.

In the illustrated embodiment, the valve member 830 is seated within the receptacle 178 of the housing 150. The valve member 830 can be seated concentrically within the receptacle 178 such that the outer edge 837 of the distal face 836 of the valve member 830 aligns, or fits concentrically, with the circular base 185 of the inner cavity wall 182. As seen, the distal face 836 extends radially inward and distal, within the receptacle 178 formed by the valve member engagement portion 183 (e.g., the first conical portion 184 and the second conical portion 186).

As seen, the extension portion 840 can extend proximally from the proximal portion 832 and connect to the anchoring member 842.

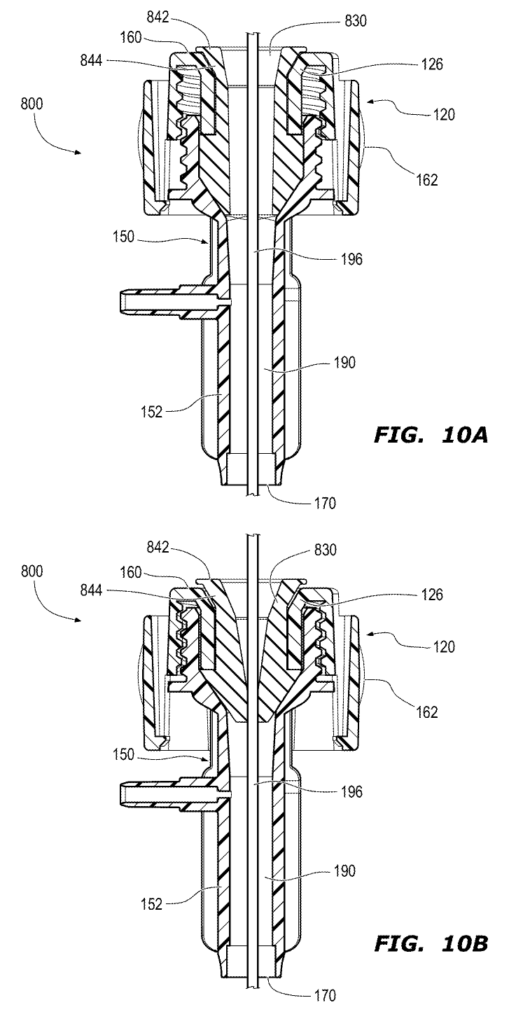

FIGS. 10A-B illustrate lateral, cutaway views of the valve member 830 seated within the housing 150 of the compression valve 110 of FIG. 2, and will be described in tandem. Accordingly, FIG. 10A illustrates a lateral, cutaway view of the compression valve 110 of FIG. 2 with the valve member 830 in an open configuration, according to embodiments described herein. FIG. 10B illustrates a lateral, cutaway view of an additional embodiment of the compression valve 110 of FIG. 2 with the valve member 830 in a closed configuration, according to embodiments described herein.

As illustrated, when transitioning from the closed configuration seen in FIG. 10B, to the open configuration seen in FIG. 10A, face 844 of the anchoring member 842 (and/or extension portion 840) can engage with the inner wall 126 of the compression member 120 to fix or the valve member 830 to the compression member 120. For instance, in some embodiments, the compression valve 800 of FIG. 10B can be transitioned from the closed configuration to the open configuration by rotating the rotating portion 162 such that the compression member 120 is retracted with respect to the housing 150. In such cases, enhanced fixation of the valve member 830 to the compression member 120 can aid in retracting the valve member 830, proximally from within the lumen 190 of the housing 150. Such retraction can enhance the disengagement or the seal formed by the valve around one (or more) elongate members.

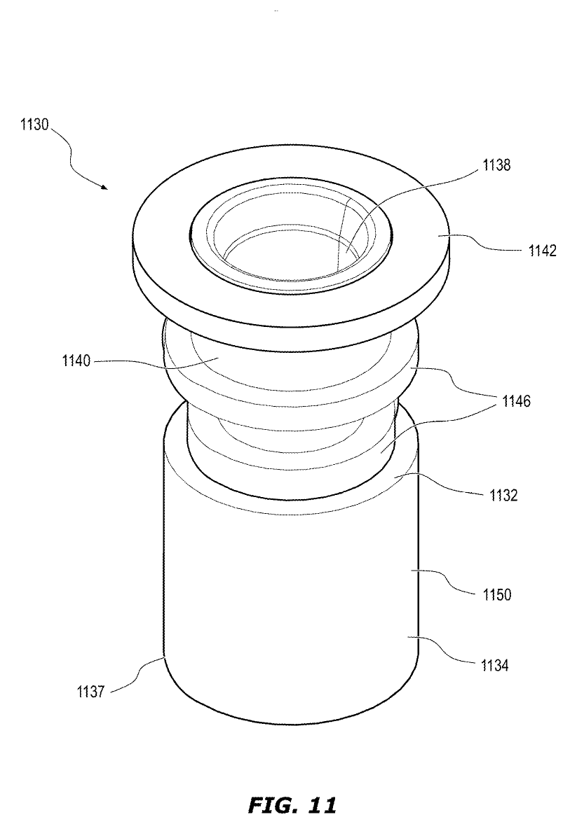

FIG. 11 illustrates a perspective view of an embodiment of a valve member 1130, according to embodiments described herein. As seen in the illustrated embodiment, the valve member 1130 includes a sealing portion 1150, an extension portion 1140, and an anchoring member 1142. The sealing portion 1150 can include a proximal portion 1132 and an outer wall 1134 extending from the proximal portion 1132 to an outer edge 1137 of a distal face. A lumen 1138 can extend through the sealing portion 1150 (and the valve member 1130 at large). In some embodiments, the lumen 1138 can form a portion of the passageway 124 of the compression valve 110.

The extension portion 1140 can extend from the proximal portion 1132 and connect to the anchoring member 1142. A face or surface of the anchoring member 1142 can be configured to engage with a portion of the compression member 120, and anchor, or fix, the valve member 1130 to the compression member 120 of the compression valve 110. Additionally, the extension portion 1140 can include ridges 1146 to enhance fixation to the compression member 120.

In some embodiments, the valve member 1130 can be formed from separate pieces. For instance, a sealant or adhesive can be used to join the sealing portion 1150, the extension portion 1140, and the anchoring member 1142 of the valve member 1130 to each other. In some embodiments, the extension portion 1140 can form an interlock to fix to the sealing portion 1150 and/or anchoring member 1142. In some embodiments, the valve member 1130 can be a monolithic piece.

In some embodiments, the valve member 1130 may be formed as one, monolithic piece. In some embodiments, the valve member 1130 can include more than one ridges 1146 for securing to the compression member 120.

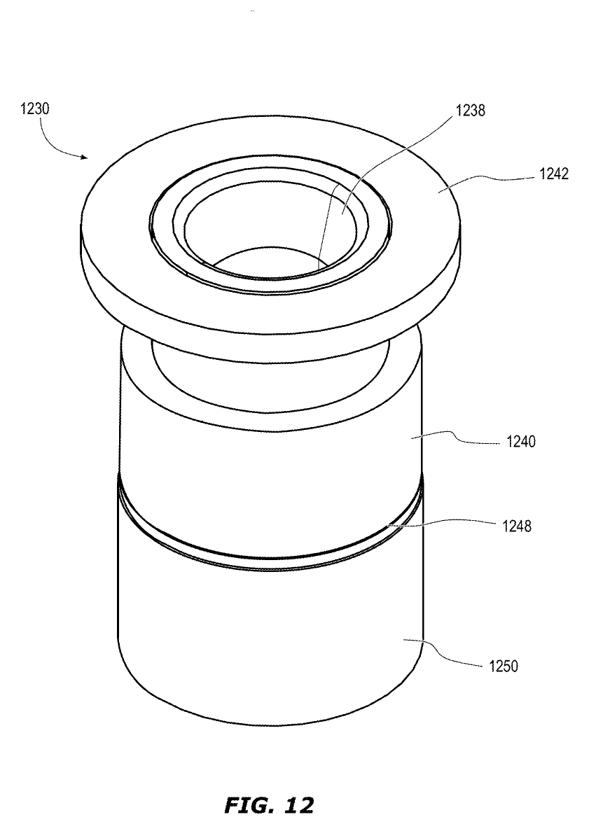

FIG. 12 illustrates a perspective view of an embodiment of a valve member 1230, according to embodiments described herein. In the illustrated embodiment, the valve member 1230 includes a sealing portion 1250, an extension portion 1240, and an anchoring member 1242. The valve member 1230 can include a spacer 1248 for joining the sealing portion 1250 and the extension portion 1240.

In some embodiments, the spacer 1248 can introduce a cavity or void by not extending to the lumen 1238. In some cases, the spacer 1248 may include a void or cavity. A sealant or adhesive can be introduced into, or around, the cavity formed by the spacer 1248, the extension portion 1240, and/or the sealing portion 1250 of the valve member 1230. The sealant can bind together the distinct portions of the valve member 1230. In alternate embodiments the valve member 1230 can be a monolithic piece.

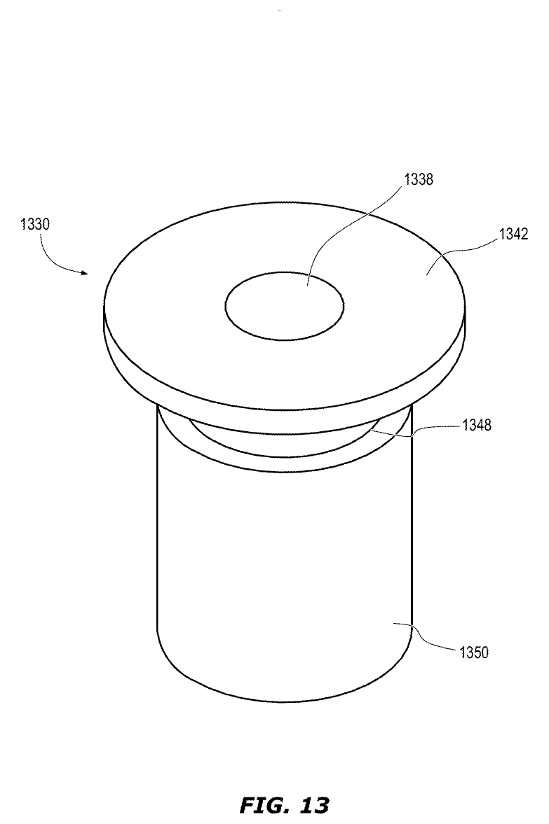

FIG. 13 illustrates a perspective view of an embodiment of a valve member 1330, according to embodiments described herein. In the illustrated embodiment, the valve member 1330 includes a sealing portion 1350, a divot 1348, and an anchoring member 1342. In some cases, the valve member 1330 may be formed as one monolithic piece.

When assembled within the compression valve 110, the inner wall 126 of the compression member 120 can intrude, or engage, with the divot 1348 of the valve member 1330. When transitioning from the closed configuration to the open configuration, the divot 1348 of the valve member 1330 can engage with the inner wall 126 of the compression member 120 to fix or anchor the valve member 1330 to the compression member 120.

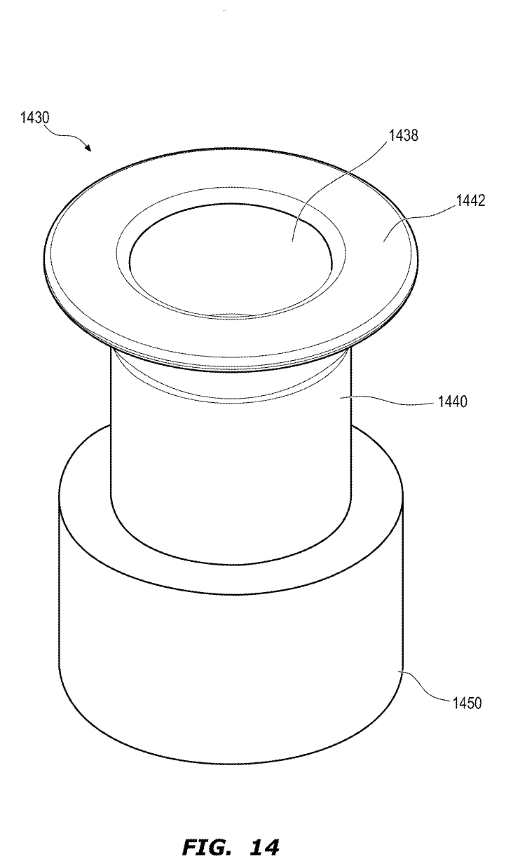

FIG. 14 illustrates a perspective view of an embodiment of a valve member 1430, according to embodiments described herein. In the illustrated embodiment, the valve member 1430 includes a sealing portion 1450, an extension portion 1440, and an anchoring member 1442. In some embodiments, the valve member 1730 may be formed as one monolithic piece.

In some embodiments, the inner wall 126 of the compression member 120 can intrude, or engage, with the extension portion 1440 (and/or a proximal face of the sealing portion 1450, and/or a distal face of the anchoring member 1442) of the valve member 1430. When transitioning from the closed configuration to the open configuration, the valve member 1430 can engage with the inner wall 126 of the compression member 120 to further fix, or anchor, the valve member 1430 to a compression member 120.

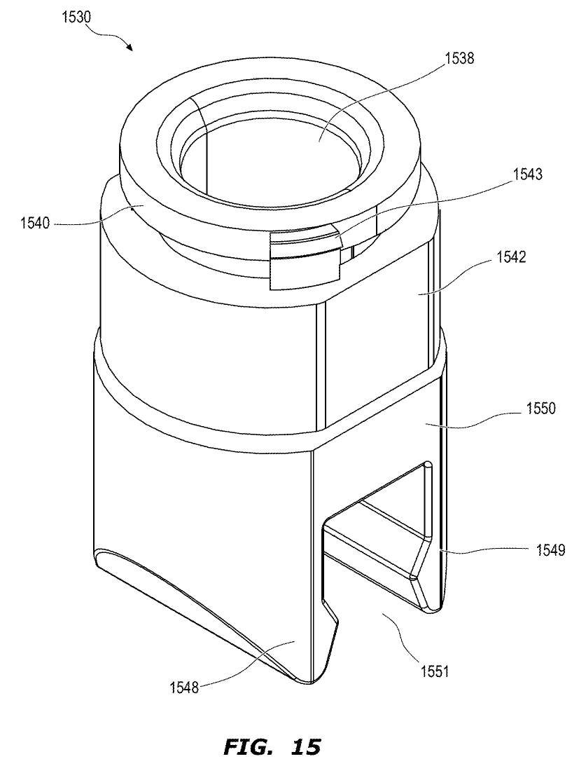

FIG. 15 illustrates a perspective view of an additional of a valve member 1530, according to embodiments described herein. In the illustrated embodiment, the valve member 1530 includes a sealing portion 1550, an extension portion 1540, and an anchoring member 1542. The sealing portion 1550 can include the sealing arms 1548, 1549 which can include, or define, an interior cavity or gap 1551. The sealing arms 1548 may be configured to bend and clamp together. A cross-section of gap 1551 can be rectangular, and an interior wall of the sealing arms 1548, 1549 can define a gap 1551.

The anchoring member 1542 can be configured to attach or fix to extension portion 1540. For instance, the anchoring member 1542 can fixate around an exterior circumferential surface of the extension portion 1540. The anchoring member 1542 can include one or more anchors 1543 around an exterior circumference, which can be configured to fix the anchoring member 1542 to the extension portion of the valve member 1530.

In some embodiments, the sealing arms 1548, 1549 can be compressed against a valve member engagement portion of a housing. When compressed against an engagement portion, the sealing arms 1548, 1549 can bend inwardly, or towards each other. Otherwise stated, the sealing arms 1548, 1549 can close the gap 1551 defined between the sealing arms 1548, 1549. When sufficiently closed (e.g., within a closed configuration), the sealing arms 1548, 1549 can collapse around a guidewire (or more than one), or an elongate member (or more than one) passing through the device, to form a seal.

In alternate embodiments, the valve member 1530 may be formed as one monolithic piece.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated by one of skill in the art with the benefit of this disclosure that in the above description of embodiments, various features are sometimes grouped together in a single embodiment, figure, or description thereof for the purpose of streamlining the disclosure. This method of disclosure, however, is not to be interpreted as reflecting an intention that any claim requires more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. It will be apparent to those having skill in the art that changes may be made to the details of the above-described embodiments without departing from the underlying principles of the present disclosure.

We claim:

1. A compression valve, comprising:
a housing comprising an engagement portion including a cross-sectional area that is substantially elliptical or rectangular;
a valve member disposed within the housing, the valve member comprising a lumen; and
a compression member proximally attached to the housing;
wherein the compression member is configured to compress the valve member against the engagement portion such that a cross-sectional area of the lumen of the valve member is substantially elliptical or rectangular.

2. The compression valve of claim 1, wherein the engagement portion comprises a substantially conical surface.

3. The compression valve of claim 1, wherein the cross-sectional area of the lumen of the valve member is substantially circular when the valve member is in an uncompressed state.

4. The compression valve of claim 1, wherein the compression member is configured to compress the valve member against the engagement portion such that the cross-sectional area of the lumen of the valve member is substantially diminished.

5. The compression valve of claim 1, wherein the compression member is configured to compress the valve member against the engagement portion such that a seal is formed between the valve member and an elongate member disposed within the lumen of the valve member.

6. The compression valve of claim 1, wherein the compression member is configured to compress the valve member against the engagement portion such that a seal is formed between the valve member, a first elongate member disposed within the lumen of the valve member, and a second elongate member disposed within the lumen of the valve member.

7. A compression valve, comprising:
a housing comprising an engagement portion comprising a major diameter and a minor diameter;
a valve member disposed within the housing, the valve member comprising a lumen; and
a compression member proximally attached to the housing;
wherein the compression member is configured to compress the valve member against the engagement portion such that a cross-sectional area of the lumen of the valve member comprises a major diameter and a minor diameter.

8. The compression valve of claim 7, wherein a ratio of the major diameter of the engagement portion to the minor diameter of the engagement portion increases along a direction from a proximal end of the engagement portion to a distal end of the engagement portion.

9. The compression valve of claim 7, wherein a cross-sectional area of the engagement portion diminishes along a direction from a proximal end of the engagement portion to a distal end of the engagement portion.

10. The compression valve of claim 7, wherein the engagement portion comprises a substantially conical surface.

11. The compression valve of claim 7, wherein the cross-sectional area of the lumen of the valve member is substantially circular when the valve member is in an uncompressed state.

12. The compression valve of claim 7, wherein the compression member is configured to compress the valve member against the engagement portion such that a seal is formed between the valve member and an elongate member disposed within the lumen of the valve member.

13. The compression valve of claim 7, wherein the compression member is configured to compress the valve member against the engagement portion such that a seal is formed between the valve member, a first elongate member disposed within the lumen of the valve member, and a second elongate member disposed within the lumen of the valve member.

14. A compression valve, comprising:
a housing comprising an engagement portion;
a valve member disposed within the housing, the valve member comprising a lumen; and
a compression member proximally attached to the housing;
wherein the compression member is configured to compress the valve member against the engagement portion such that a cross-sectional area of the lumen of the valve member transitions from an initial shape to an intermediate shape, and from the intermediate shape to a final shape.

15. The compression valve of claim 14, wherein the initial shape comprises an aspect ratio that is less than an aspect ratio of the intermediate shape.

16. The compression valve of claim 14, wherein the initial shape is substantially circular.

17. The compression valve of claim 14, wherein the intermediate shape is substantially elliptical.

18. The compression valve of claim 14, wherein the final shape is substantially rectangular.

19. The compression valve of claim 14, wherein the valve member further comprises:
a first arm and a second arm disposed defining the lumen of the valve member;
wherein compression of the valve member against the engagement portion is configured to diminish a distance between the first and second arms such that the cross-sectional area of the lumen of the valve member is diminished.

20. The compression valve of claim 14, wherein the initial shape is substantially rectangular.
